(19) United States
(12) Patent Application Publication
(10) Pub. No.: US 2022/0315677 A1
(43) Pub. Date: Oct. 6, 2022

(54) Metallocene Catalyzed Propylene Oligomerisation Without Separation of Alkane and Olefin

(71) Applicant: CHEVRON U.S.A. INC., San Ramon, CA (US)

(72) Inventor: Sven Ivar Hommeltoft, Pleasant Hill, CA (US)

(21) Appl. No.: 17/709,579

(22) Filed: Mar. 31, 2022

Related U.S. Application Data
(60) Provisional application No. 63/169,209, filed on Apr. 1, 2021.

(51) Int. Cl. C08F 110/06; C07C 4/06; C07C 2/32; B01D 53/04

(52) U.S. Cl. CPC C08F 110/06; C07C 4/06; C07C 2/32; B01D 53/04; C08F 2420/00; C07C 2531/14; C07C 2531/22; B01D 2253/104

(57) ABSTRACT

The present invention is directed to metallocene catalyzed oligomerization of olefin feed stock without fractionation of alkane and olefin.

### FIELD OF THE INVENTION

This invention relates to metallocene catalyzed propylene oligomerization of refinery olefin feed stock.

### BACKGROUND 10 OF THE INVENTION

Olefins feedstock used in metallocene catalysis are typically purified to very high levels of purity or “polymer grade” for several reasons.

An Important reason for this is that the metallocene catalysis is highly sensitive to impurities in the feedstock that could passivate the metallocene catalysis. Another reason for the high olefin purity is that the oligomerisation reaction produces lighter products and the reaction kinetics are typically slowed when the olefin is diluted with even moderate amounts of saturated alkanes that are otherwise inert in the oligomerisation chemistry.

In some applications lighter products are desired or at least acceptable. It has been previously proposed to add inert alkanes as diluents or solvents to aid in controlling the reaction exotherm through dilution and through slowing down the kinetics. However, in order to control the levels of impurities that could passivate the metallocene catalyst complexes refinery olefin feedstock, that almost always contains high levels of catalyst passivation impurities are typically fractionated to a high purity levels to eliminate most of these impurities and subsequently diluted with inert diluents or solvents. The separation of olefins and alkanes such as for instance separation of propylene and propane that have very narrow boiling point differences is a very costly fractionation and it would therefore be economically beneficial if this fractionation step could be avoided.

### SUMMARY OF THE INVENTION

An embodiment of the invention is a method to oligomerize refinery olefin feed stock over metallocene catalysis without the addition and subsequent separation of alkane.

Another embodiment is the use of absorption steps to remove the impurities in the refinery olefin streams to sufficiently low levels to allow metallocene catalysis to convert the refinery olefins without separation of alkane and olefin and without excessive use of metallocene catalyst complexes and activator.

### DETAILED DESCRIPTION OF THE INVENTION

Herein is described a method to oligomerize refinery olefin feed stock over metallocene catalysis without the separation of alkane and olefin. A preferred embodiment is the use of absorption steps to remove the impurities in the refinery olefin streams to sufficiently low levels to allow metallocene catalysis to convert the refinery olefins without the expensive fractionation of alkane and olefin and without excessive use of metallocene catalyst complexes.

Dienes may be included in oligomerisation with some metallocene catalyst but for many metallocene catalyst dienes act as catalyst passivators and the oligomerisation process in those cases requires that they be removed down to below 100 ppm level or lower.

The term “impurities” as used herein describes substance that would act as a catalyst passivator.

The term “oligomerisation reactor” as used herein describes an apparatus for oligomerization of lower olefins to produce higher olefins. In this context, “lower” and “higher” are relative; a lower alpha-olefin is converted to a higher olefin, or higher alpha-olefin, having a greater number of carbon atoms. The reaction is carried out in the presence of one or more catalyst systems under conditions encouraging the reaction to proceed. As described herein at least 90% of the propylene present in the olefin containing feed is converted to oligomers and an amount of hydrogen corresponding to 0.01-5 mole % relative to total amount of olefin in the olefin containing stream is present in the oligomerization reactor.

An embodiment of the process is metallocene propylene oligomerisation without fractionation of olefin and alkane comprising the steps of:

(a) Optionally treating an olefin containing feed with an aqueous caustic solution,

(b) drying over a molecular sieve to remove moisture and other impurities,

(c) using an absorption agent to remove impurities in the olefin containing feed,

(d) feeding product of step (c) to a metallocene catalysis reactor to convert the olefins without fractionation of alkane and olefin.

A further embodiment is a process for producing propylene oligomers from a hydrocarbon feedstock comprising the steps of:

a. Cracking the hydrocarbon feedstock to form a cracked product, wherein the cracking may comprise coking, catalytic cracking and fluid catalytic cracking,

b. Isolating from the cracked product stream an olefin containing stream containing no less than 70% C3 hydrocarbons, propane being 8-35% by weight of the C3 hydrocarbons, and propylene being 65-92% by weight of the of the C3 hydrocarbons,

c. optionally treating said olefin containing stream with an aqueous caustic solution,

d. drying over a molecular sieve to remove moisture and other impurities

e. using an absorption agent to remove remaining traces of impurities in the olefin containing stream,

f. feeding the olefin containing stream without separating propane and propylene to at least a first oligomerisation reactor operating at a temperature of 20-100° C. and a pressure sufficient to keep the reaction mixture substantially in the liquid phase, in which the olefins are oligomerized in the presence of a metallocene catalyst to form olefin oligomers, converting at least 50% of the propylene present in the olefin containing feed,

g. withdrawing an oligomer rich reactor effluent from the oligomerization reactor and isolating an oligomer product.

A preferred embodiment is hydrocarbon feedstock that has a flash point higher than 100° C., a T10 higher than 200° C., T50 higher than 350° C. Additionally, the hydrocarbon feedstock comprises propane and the cracking is a dehydrogenation reaction in which propane is dehydrogenated to form propylene.

The absorption agent may be an activated alumina wherein a preferred embodiment is the use of activated alumina selected from the group consisting of Selexsorb COS and Selexsorb CD.

Contact time for absorbent and feed is about 10 minutes to at least 10 hours.

While the invention is applicable to any combination of light refinery streams rich in C2-C5 olefins including FCC olefins and Coker olefins, propylene rich streams such as the C3 cut from an FCC stream is particularly suitable because of the low content of dienes and other impurities in the C3 stream. Butenes and pentenes from FCC or Coker operations contain significant amounts of dienes that can challenge some metallocene catalysts and result in prohibitive catalyst passivation.

In one embodiment the refinery olefins stream, typically from an FCC or a Coker unit, such as an FCC C3 stream treated with aqueous caustic, dried over a molecular sieve such as 13× sieve to remove moisture and other impurities and finally passed over activated alumina to remove the last traces of impurities before being fed to the oligomerisation reactor and a purified olefin/alkane mixture such as for instance a propylene propane mixture. The optimal choice of activated alumina depends on the impurities in the olefin feed streams but Selexsorb COS or Selexsorb CD may be used. In another embodiment, the caustic wash of the olefin stream is omitted.

The metallocene catalyst may be prepared by interaction of a metallocene catalyst precursor with an activator with methods known to one of skill in the art.

A preferred embodiment for formation of the metallocene catalyst is by interaction with an activator from an unbridged metallocene complex of the general formular Cp,Cp′ MX2, (Cp, Cp′ are cyclopentadienyl groups with one or more substituents on the cyclopentadienyl rings these substituents being selected from the group consisting of hydrogen, methyl, ethyl, isopropyl, n-propyl, n-butyl, tert-butyl, sec-butyl, C5-C16 n-alkyl, C5-C10 isoalkyl, M is Zr or Hf, and X is Cl or Br. The metallocene catalyst precursor may be a complex of the general formula (R,R′Cp)2MXY where RR′Cp are mono or poly substituted cylopentadienyl groups or mono or polysubstituted indenyl groups, M is a metal selected from the group of Ti, Zr or Hf and X and y are chosen from the group of (Cl, Br, I, H, Me, alkyl).

The activator may be any reactant or combination of reactants that by reaction with the metallocene catalyst precursor complex can convert it into a form capable of catalyzing olefin oligomerisation. Examples of activators are methylaluminoxane (MAO) or modified methylaluminoxane (MMAO) in which some fraction of the methyl groups in methylaluminoxane has been replaced by other alkyl groups. strong Lewis acids such as trisperfluorophenyl borane or other trisubstituted boranes with strongly electron withdrawing substituents. Another activator is a combination of tri-alkylaluminum (R,R′,R″Al, R, R′, R″ chosen from H, Me, Alkyl) combine with a solid super acid such as for instance fluorinated alumina.

### Example 1

Conversion of refinery propylene to oligomers in a continuously operated oligomerisation unit.

This experiment was done using a continuously operating loop reactor unit centered on a loop reactor with a reactor volume of approximately 600 ml. Then temperature of the reactor loop was controlled by submersion of a large part of the loop into a thermostat bath. The pressure in the reactor loop was maintained at around 620-650 psi. The olefin feed stream used was a refinery FCC PP mixture containing about 70% propylene and about 30% propane. This olefin feed stream was pumped at a flow rate of 2.7 ml/min through a 13× molecular sieve absorber and then through a second absorber filled with Selexsorb CD activated alumina before being introduced into the reactor loop. A hydrogen stream of 5.7 nmL/min administered by a mass flow controller was injected into the olefin stream upstream of the reactor loop. The metallocene catalyst was introduced in form of a solution containing a 9:1 mixture of (tBuCp)2ZrCl2 and (i-PrCp)2ZrCl2 (Zr concentration: 0.02 wt % or 0.0042 mmole/ml) and MMAO-20 (Al concentration; 2.57 wt %) in a non-olefinic hydrocarbon solution. The catalyst solution was introduced directly into the reactor loop at a flow of 0.02 ml/min. The reactor effluent was depressurized downstream of the reactor and propane and unconverted propene allowed to escape into the vent leaving an oligomer product (0.43-0.44 g/min).

T1H NMR of the oligomer products showed an olefin content corresponding to an average chainlength around 25-30 carbons.

The SIMDIS of the different samples show: T10=450-460 F, T30=700-780, T50=860-1000 F, T70=1050-1190, T90=1220-1340 F, End point: >1400 F for most sample.

What is claimed is:

1. A process producing propylene oligomers from a hydrocarbon feedstock comprising the steps of:
a. Cracking the hydrocarbon feedstock to form 5 a cracked product,
b. Isolating from the cracked product stream an olefin containing stream containing no less than 70% C3 hydrocarbons, propane being 8-35% by weight of the C3 hydrocarbons, and propylene being 65-92% by weight of the of the C3 hydrocarbons,
c. optionally treating said olefin containing stream with an aqueous caustic solution,
d. drying over a molecular sieve to remove moisture and other impurities,
e. using an absorption agent to remove remaining traces of impurities in the olefin containing stream,
f. feeding the olefin containing stream without separating propane and propylene to at least a first oligomerisation reactor operating at a temperature of 20-100° C. and a pressure sufficient to keep the reaction mixture substantially in the liquid phase, in which the olefins are oligomerized in the presence of a metallocene catalyst to form olefin oligomers, converting at least 50% of the propylene present in the olefin containing feed,
g. withdrawing an oligomer rich reactor effluent from the oligomerization reactor and isolating an oligomer product.

2. Process of claim 1, wherein the hydrocarbon feedstock has a flash point higher than 100° C., a T10 higher than 200° C., T50 higher than 350° C.

3. Process of claim 2, where in the cracking is catalytic cracking.

4. Process of claim 3, wherein the catalytic cracking is fluid catalytic cracking.

5. Process of claim 1, wherein the cracking comprises coking.

6. Process of claim 1, wherein the hydrocarbon feedstock comprises propane and the cracking is a dehydrogenation reaction in which propane is dehydrogenated to form propylene.

7. The process of claim 1, wherein the absorption agent is an activated alumina.

8. The process of claim 7, wherein the activated alumina is selected from the group consisting of Selexsorb COS and Selexsorb CD.

9. The process of claim 1, wherein the metallocene catalyst is formed by interaction with an activator from an unbridged metallocene complex of the general formular Cp,Cp′ MX2, (Cp, Cp′ are cyclopentadienyl groups with one or more substituents on the cyclopentadienyl rings these substituents being selected from the group consisting of hydrogen, methyl, ethyl, isopropyl, n-propyl, n-butyl, tert-butyl, sec-butyl, C5-C16 n-alkyl, C5-C10 isoalkyl, 5 M is Zr or Hf, and X is Cl or Br.

10. The process of claim 9 wherein the activator is methylaluminoxane. (MAO) or modified methylaluminoxane (MMAO) in which some fraction of the methyl groups in methylaluminoxane has been replaced by other alkyl groups.

11. Process of claim 1, an amount of hydrogen corresponding to 0.01-5 mole % relative to total amount of olefin in the olefin containing stream is present in the oligomerization reactor.

12. The process of claim 1 wherein at least 90% of the propylene present in the olefin containing feed is converted to oligomers.
